(19) United States
(12) Patent Application Publication
(10) Pub. No.: US 2024/0182514 A1
(43) Pub. Date: Jun. 6, 2024

(54) Process for the Preparation of Alfaxalone

(71) Applicant: Respect Labs LLP, Navi Mumbai (IN)

(72) Inventors: Kodali Eswara Rao, Navi Mumbai (IN); Bobba Venkata Sivakumar, Navi Mumbai (IN)

(21) Appl. No.: 18/282,191

(22) PCT Filed: Mar. 16, 2022
(86) PCT No.: PCT/IN2022/050254

§ 371 Date: Sep. 14, 2023

(30) Foreign Application Priority Data
Mar. 16, 2021 (IN) 202121011214

(51) Int. Cl. C07J 7/00

(52) U.S. Cl. CPC C07J 7/002

(57) ABSTRACT

The present invention provides an improved process for the preparation of Alfaxalone compound of structural Formula I and, which is simple, ecofriendly, cost effective and commercially viable. More particularly the present invention provides a process for preparation of compound of structural Formula I by reacting compound of structural Formula III with a reducing agent to give a mixture of diastereoisomeric compound of structural Formula II. Then separating diastereoisomeric compound of structural Formula II by chromatographic technique to get compound of structural Formula I.

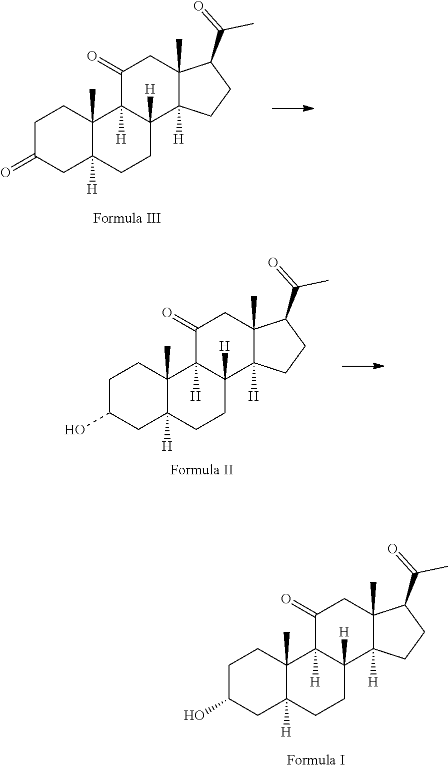

### FIELD OF THE INVENTION

The present invention relates to an improved process for preparation of Alfaxalone compound of structural Formula I, which is simple, ecofriendly, cost effective and commercially viable. More particularly the present invention provides a process for preparation of compound of structural Formula I by reacting compound of structural Formula III with a reducing agent to give a mixture of diastereoisomeric compound of structural Formula II. Then separating diastereoisomeric compound of structural Formula II by chromatographic technique to get compound of structural Formula I.

### BACKGROUND OF THE INVENTION

Alfaxalone is chemically described as 3-α-hydroxy-5-α-pregnane-11, 20-dione, is a potent neuroactive steroid anaesthetic currently used in veterinary medicine (Child et al., British Journal of Anaesthesia 43:2-13, 1971) and has a molecular weight 332.5. Alfaxalone compound of structural Formula I has the following chemical structure:

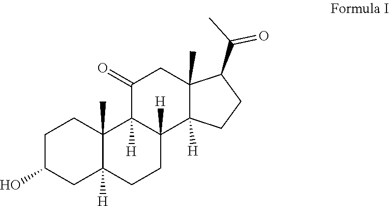

The primary mechanism for the anesthetic action of Alfaxalone is modulation of neuronal cell membrane chloride ion transport, induced by binding of Alfaxalone to GABA (gammaaminobutyric acid) cell surface receptors.

Alfaxalone was widely used around the world as an intravenous anesthetic. Alfaxalone was approved as Alfaxalone injectable solution (10 mg/ml) in USA under trademark ALFAXAN® MULTIDOSE on Sep. 6, 2012 to Jurox Pty. Ltd.

Alfaxalone (ALFAXAN® MULTIDOSE) injectable solution (10 mg/ml) is indicated for use in:
a) Dogs for the treatment of:
1) Induction of general anesthesia.
2) Maintenance of general anesthesia following induction.
3) For induction of anesthesia followed by maintenance with an inhalant anesthetic.
b) Cats for the treatment of:
1) Induction of general anesthesia.
2) For induction of anesthesia followed by maintenance with an inhalant anesthetic.
3) Maintenance of general anesthesia following induction.

Although these anesthetics have a high therapeutic index, they were nevertheless withdrawn from clinical practice due to occasional, unpredictable yet severe anaphylactoid reactions to the polyethoxylated castor oil excipient (Cremophor EL) which formed part of the formulation for administration.

Alfaxalone is previously known from the U.S. Pat. No. 3,714,352. The said patent discloses a method of inducing anaesthesia in an individual man and animal, it also discloses the preparation of Alfaxalone compound of structural Formula I as shown below in scheme I.

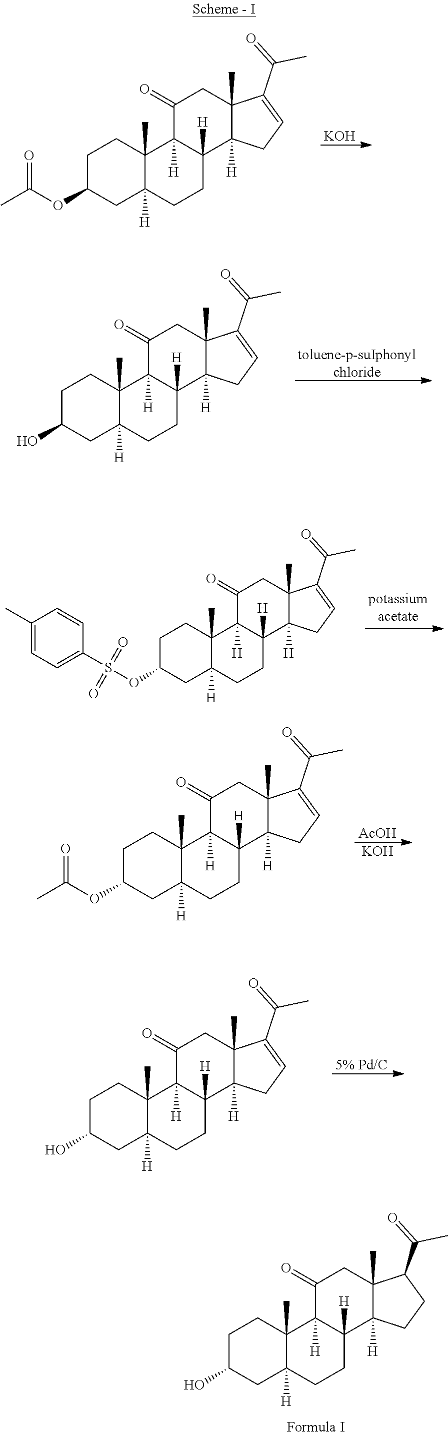

The above process for the preparation of compound of structural Formula I disclosed in U.S. Pat. No. 3,714,352, suffer from low yields, lack of selectivity, require extensive purification and/or not readily scalable.

Journal of the American Chemical Society 1953, 75, 1286-1290 by Mancera, O.; Ringold, H. J.; Djerassi, C.; Rosenkranz, G.; Sondheimer, F. A ten-step conversion of progesterone to cortisone. The differential reduction of pregnane-3,11,20-trione with sodium borohydride, discloses the differential reduction of the 3-keto group to yield the important cortisone intermediate pregnan-3α-ol-11,20-dione was carried out with the low conversion rate by means of sodium borohydride in pyridine solution which is comparatively low with the process of present invention.

WO2020006596 discloses process for the preparation of Alfaxalone compound of the structural Formula I, as shown below scheme II.

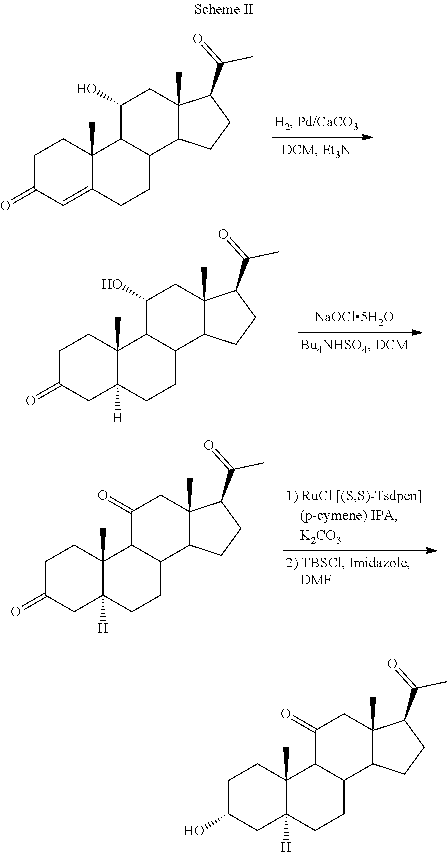 [SMILES: CC(=O)[C@H]1CCC2C3CCC4=CC(=O)CC[C@]4(C)C3[C@H](O)C[C@@]21C ; CC(=O)[C@H]1CCC2C3CC[C@H]4CC(=O)CC[C@]4(C)C3[C@H](O)C[C@@]21C ; CC(=O)[C@H]1CCC2C3CC[C@H]4CC(=O)CC[C@]4(C)C3C(=O)C[C@@]21C ; CC(=O)[C@H]1CCC2C3CC[C@H]4C[C@H](O)CC[C@]4(C)C3C(=O)C[C@@]21C]

The above prior art processes for the synthesis of Alfaxalone compound of the structural Formula I, are not commercially viable due to low yield, lack of selectivity, expensive purification techniques.

Helvetica Chimica Acta, 2011, Vol. 94, pages 98-104 by Zhang, Zonglei et. al., “First Synthesis of a C-Homosteroid from Pregn-4-ene-3,11,20-trione” discloses the first synthesis of a C-homosteroid from pregn-4-ene-3,11,20-trione by the selective reduction of the 3-oxo group of (5α)-pregnane-3,11,20-trione to give the 3α-hydroxy steroid using K-Selectride in dry THF. However, the process disclosed in this prior art is difficult to operate due to abnormal reaction temperature condition of “−78° C.”. Further, this prior art does not disclose detailed working experimental conditions and example, quantities of raw materials, details of reaction conditions and purity data for final product.

There is still need in the art to develop a simple, ecofriendly, cost effective and commercially viable process for the preparation of Alfaxalone compound of structural Formula I.

The inventors of the present invention surprisingly found a regioselective and improved process for the preparation of Alfaxalone compound of structural Formula I, which is simple, ecofriendly, cost effective and commercially viable.

### SUMMARY OF THE INVENTION

Accordingly, the present invention is to provide a process for preparation of Alfaxalone compound of structural Formula I.

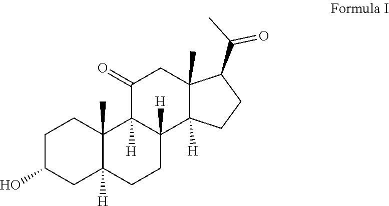

In another aspect of the present invention is to provide a process for preparation of compound of structural Formula I, comprising steps of:
(a) reacting compound of structural Formula III with a reducing agent to give a mixture of diastereoisomeric compound of structural Formula II in an organic solvent at a suitable temperature condition

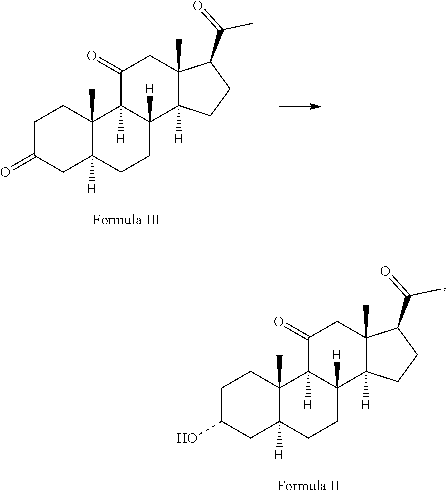

(b) separating diastereoisomeric compound of structural Formula II by chromatographic technique to get compound of structural Formula I

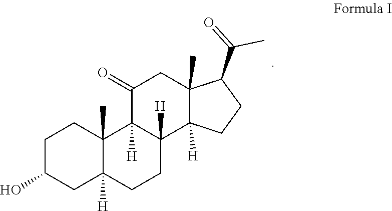

In another aspect of the present invention is to provide a process for preparation of compound of structural Formula I, comprising steps of:
(a) reacting compound of structural Formula III with organoboron compounds to give a mixture of diastereoisomeric compound of structural Formula II in organic solvent at a suitable temperature condition

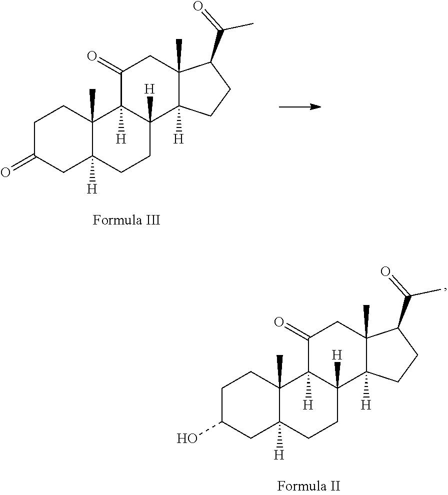

(b) separating diastereoisomeric compound of structural Formula II by chromatographic technique to get compound of structural formula I

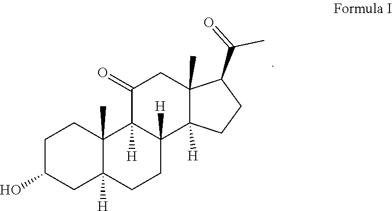

In another aspect of the present invention is to provide a process of preparation of compound of structural Formula I, comprising steps of:
(a) reacting compound of structural Formula III with (−)-diisopinocampheyl chloroborane to give a mixture of diastereoisomeric compound of structural Formula II in tetrahydrofuran at a temperature in the range of −30° C. to 50° C.

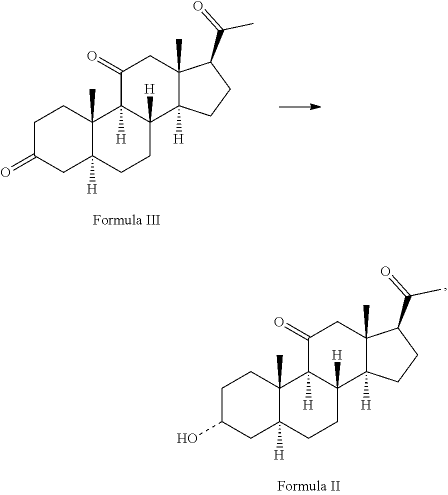

(b) separating diastereoisomeric compound of structural Formula II by chromatographic technique to get compound of structural formula I

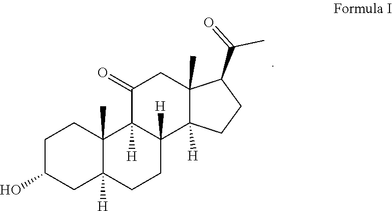

In another aspect of the present invention is to provide a process for preparation of compound of structural Formula I, comprising steps of:
(a) reacting compound of structural Formula III with (−)-diisopinocampheyl chloroborane to give a mixture of diastereoisomeric compound of structural Formula II in tetrahydrofuran at a temperature in the range of −30° C. to 50° C.

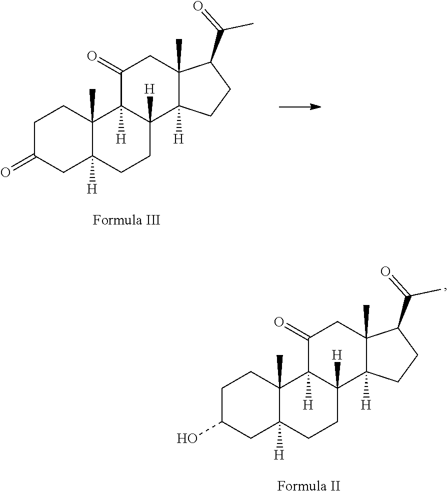

(b) separating diastereoisomeric compound of structural Formula II by chromatographic technique selected from but not limited to flash chromatography, column chromatography, liquid chromatography to get compound of structural formula I

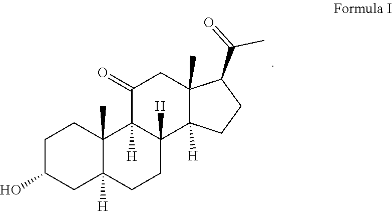

In another aspect of the present invention is to provide a process for preparation of compound of structural Formula I, comprising steps of:
(a) reacting compound of structural Formula V with an oxidizing agent to get compound of structural Formula IV

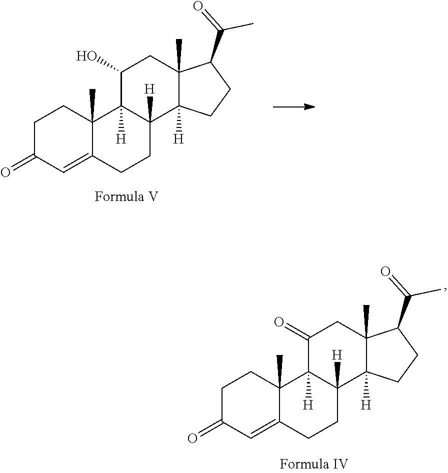

(b) reacting compound of structural Formula IV with a reducing agent to get compound of structural Formula III

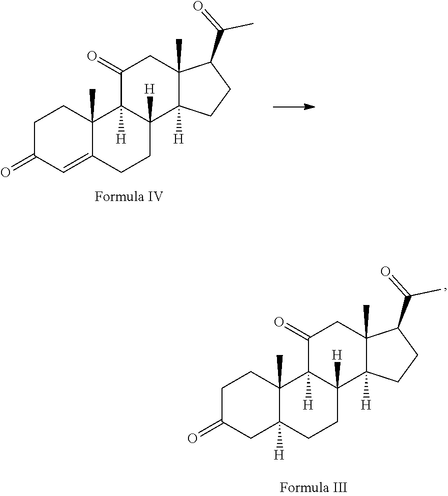

(c) reducing compound of the structural Formula III to provide compound of structure Formula I.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps of the process, features of the product, referred to or indicated in this specification, individually or collectively, and all combinations of any or more of such steps or features.

The terms and words used in the following description and claims are not limited to the bibliographical meanings but are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise.

Features that are described and/or illustrated with respect to one embodiment may be used in the same way or in a similar way in one or more other embodiments and/or in combination with or instead of the features of the other embodiments.

It should be emphasized that the term “comprises/comprising” when used in this specification is taken to specify the presence of stated features, integers, steps or components but does not preclude the presence or addition of one or more other features, integers, steps, components or groups thereof.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

The present disclosure is not to be limited in scope by the specific embodiments described herein, which are intended for the purposes of exemplification only. Functionally equivalent products and processes are clearly within the scope of the disclosure, as described herein.

Accordingly, the present invention provides a process for preparation of Alfaxalone compound of structural Formula I, comprising the steps of:
(a) reacting compound of structural Formula III with a reducing agent to give a mixture of diastereoisomeric compound of structural Formula II in an organic solvent at a temperature in the range of −30° C. to 50° C., wherein the reducing agent is organoboron compound,

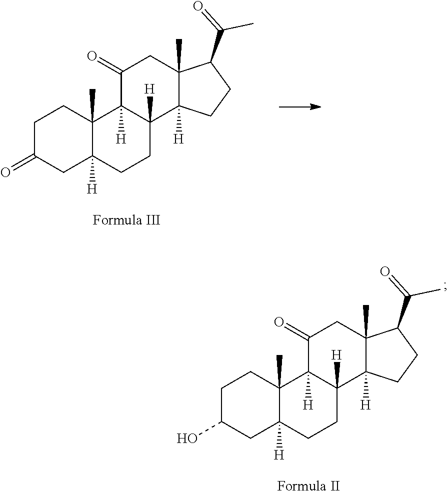

and
(b) separating diastereoisomeric compound of structural Formula II by chromatographic technique and then filtering and drying to get compound of structural Formula I:

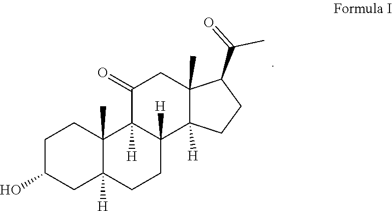

In one of the features of the present invention, the organoboron compound is selected from but not limited to (−)-diisopinocampheyl chloroborane ((−) DIP chloride), 9-Borabicyclo[3.3.1]nonane (9-BBN), Diborane. More preferably, the organoboron compound is (−)-diisopinocampheyl chloroborane.

In another feature of the present invention, the organic solvent is selected from but not limited to tetrahydrofuran (THF), Dichloromethane, Diethyl ether, Methyl tertiaiy-butyl ether (MTBE).

In yet another feature of the present invention, the step (a) is carried out at a temperature in the range of −10° C. to 0° C.

In yet another feature of the present invention, the chromatographic technique is selected from flash chromatography, column chromatography, or liquid chromatography.

In yet another feature of the present invention, the chromatographic technique is flash chromatography and in step (b) flash chromatography is used with a solvent at a mobile phase to give alfaxalone compound of structural Formula I. In one of the feature of the present invention the solvent used in flash chromatography is acetonitrile water.

In yet another feature of the present invention, the chromatographic purity of the compound of structural Formula II is 75% to 80%. In one of the features of the present invention, the chromatographic purity of the compound of structural Formula I is 99.7 to 99.9%.

In yet another feature of the present invention, the compound of structural Formula I and the compound of structural Formula II are stable for at least 6 months at room temperature.

The present invention also provides a process for preparing a Formula III, comprising the steps of:
(i) reacting compound of structural Formula V (11α-Hydroxyprogesterone) with an oxidizing agent to get compound of structural Formula IV:

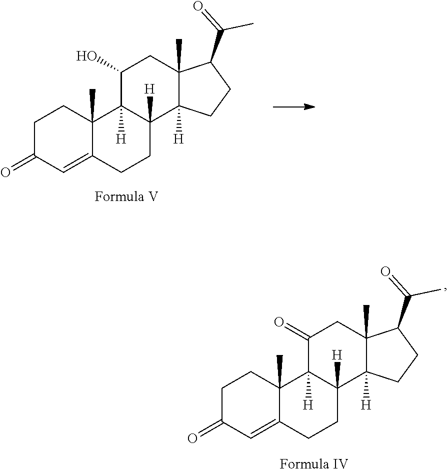

(ii) reacting compound of structural Formula IV with a reducing agent to get compound of structural Formula III:

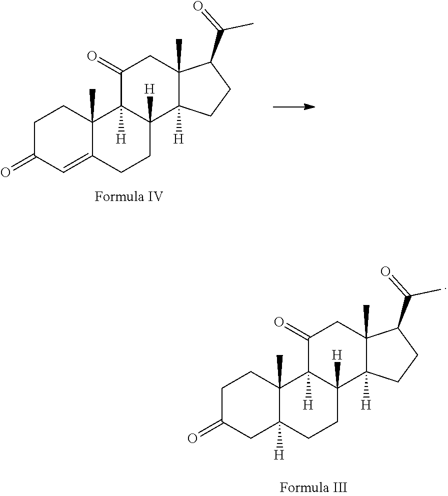

In one of the features of the present invention, the reaction of step (i) is carried out in presence of a solvent and the solvent is acetic acid and the oxidizing agent used in step (i) is selected from but not limited to chromium trioxide, Manganese (IV) oxide, pyridinium chlorochromate, sodium dichromate, potassium dichromate, DMSO-Oxalyl chloride (Swerm oxidation). More preferably the oxidizing agent is chromium trioxide.

In another feature of the present invention, the reaction of step (ii) is carried out in presence of a solvent and is selected from Dichloromethane (DCM), Isopropyl alcohol (IPA) and mixture thereof and the reducing agent is 10% Palladium on carbon (10% Pd/C).

In yet another feature of the present invention, the step (i) is carried out at a temperature in the range of 25-35° C. and the step (ii) is carried out at a temperature in the range of 50-55° C.

### Scheme and Brief Process

In one of the features of the present invention, a process for preparation of Alfaxalone compound of structural Formula I is shown in below scheme III:

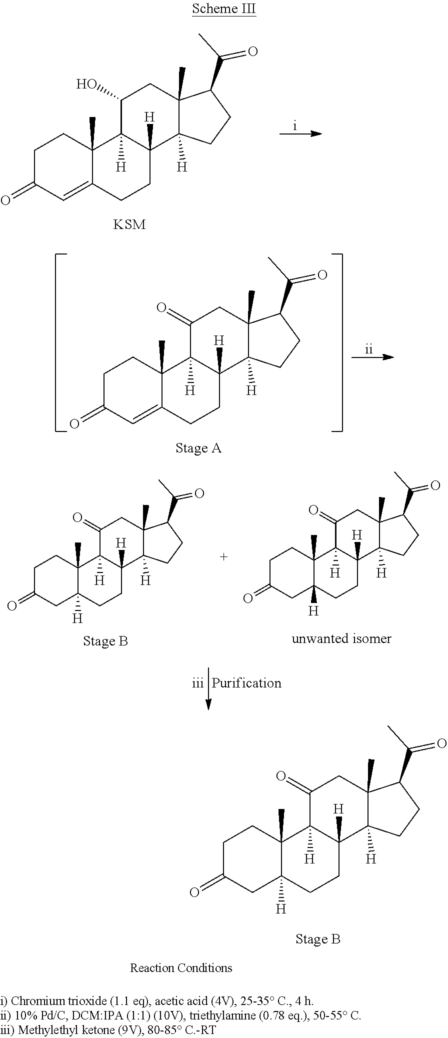

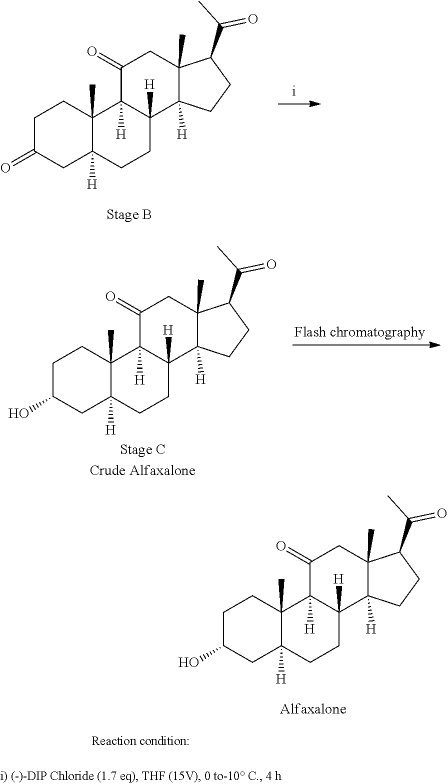

### Stage-A/Formula IV:

11α-Hydroxy progesterone is reacted with chromium trioxide in presence of acetic acid as a solvent gives stage A.

### Stage-B/Formula III:

Stage A reacted with 10% palladium content in presence of DCM & IPA as a solvent under hydrogen pressure gives Stage B.

### Stage-C/Formula II (Crude Alfaxalone):

Stage B reacted with (-) DIP chloride in presence of THF as a solvent gives crude alfaxalone (Stage C).

### Stage Final:

Crude alfaxalone purify by flash chromatography using acetonitrile water at a mobile phase gives alfaxalone compound of Formula I.

### Stereoisomerism

1) 5β-Pregnane-3,11,20-trione (CAS NO: 1474-68-6) is isomer of 5α-Pregnane-3,11,20-trione (Stage B) formed during Stage B synthesis.
2) 3β-Hydroxy-5α-pregnane-11,20-Dione (CAS NO: 600-59-9) is β-isomer of alfaxalone formed during alfaxalone synthesis.

### EXAMPLES

Additional embodiments are disclosed in further detail the following examples, which are not in any way intended to limit the scope of the claims.

### Example 1: Synthesis of Stage A/Formula IV

Charged 500 mL acetic acid at Room Temperature (RT) in 4-neck Round Bottom

Flask (RBF). Charged 125 g 11-alpha hydroxy progesterone (Formula V) at room temperature. The reaction mixture was stirred for 10-15 min at room temperature. Clear solution obtained. Slowly added chromium trioxide solution (41.6 g in 37.5 mL water) at 20° to 30° C. The reaction mixture was stirred for 5 hrs at 20° to 30° C. Slowly added 2000 mL water at 25° to 35° C. The reaction mixture was stirred at 25° to 35° C. for 1 hr. The reaction mixture was filtered and washed with 500 mL water. Charged 500 mL dichloromethane (DCM) at room temperature in 4-neck RBF. Stirred the reaction mixture for 15 min. Clear solution obtained. Organic layer was washed with 250 mL 10% sodium bicarbonate solution (3 times). Organic layer was washed with 250 mL water to obtain DCM layer containing compound of (Stage A/Formula IV).

### Example 2: Synthesis of Stage B/Formula III

Charged above DCM layer containing compound of (Stage A/Formula IV) of Example 1 at RT in autoclave. Charged 24.02 g triethylamine at RT. Charged 500 mL isopropyl alcohol at RT. Closed all valves. Flushed out autoclave with 3 Kg nitrogen gas (3 times). Flushed out autoclave with 3 Kg hydrogen gas. 10-12 kg hydrogen pressure applied. The reaction mixture was heated at 50-55° C. and maintained for 12 hr. The reaction mixture was cooled to RT. The reaction mixture was filtered through hyflow bed and washed with 200 mL Dichloromethane (DCM). Keep spend Pd/C for recovery. Arranged clean and dry 4-neck RBF. Charged clear filtrate at RT. Organic layer was washed with 500 mL 10% sodium bicarbonate solution. The organic layer was washed with 500 mL water. Distilled out organic layer u/v at 50° C. Obtained wet wt. 120-125 g. Charged 1.0 L methyl ethyl ketone at RT. The reaction mixture was heated at 80-85° C. Clear solution obtained at 80-85° C. Distilled out 5V methyl ethyl ketone at 90-95° C. The reaction mixture was cooled to RT. Stirred reaction mixture for 2 hr at 25-30° C. The reaction mixture was filtered and washed with 50 mL methyl ethyl ketone. Solid obtained was dried u/v at 50° C. for 8 hr to obtain (Stage B/Formula III). Dry wt.=52-56 g. HPLC purity NLT 92.00%.

### Example 3: Synthesis of Stage C/Formula II

Arranged clean and dry 4-neck RBF. Charged 1500 mL Tetrahydrofuran at RT. Charged 100 g compound of Stage B/Formula III at RT. The reaction mixture was stirred at RT. The reaction mixture was cooled 0° to −10° C. Slowly added 300 mL (−) DIP chloride at 0° to −10° C. The reaction mixture was stirred for 3.0 hrs at 0° to −10° C. Slowly added 50 mL methanol. The reaction mixture was stirred for 1 hr. Slowly added acetic acid solution (52 mL acetic acid in 100 ml water). The reaction mixture was stirred for 1.0 hr. The organic layer was separated. Organic layer was washed with potassium carbonate solution (125 g potassium carbonate in 500 mL water). The reaction mixture was stirred for 1.0 hr. Organic layer was separated. Organic layer was washed with sodium chloride solution (25 g sodium chloride in 500 mL water). The reaction mixture was stirred for 1.0 hrs. Organic layer was separated. Distilled out organic layer u/v at 50° C. strip out with 50 mL Heptane. Obtained oily mass 225-250 g. Charged 1000 mL DCM to the oily mass. The reaction mixture was stirred for 15 min. Organic layer was washed with Sodium bicarbonate solution (1000 mL 10% aq. sodium bicarbonate solution). The reaction mixture was stirred for 1.0 hr. The organic layer was separated. Organic layer was washed with sodium chloride solution (25 g sodium chloride in 500 mL water). The reaction mixture was stirred for 1.0 hr. Organic layer was separated. Distilled out organic layer u/v at 40° C. and strip out with 50 mL heptane. Charged 1000 mL heptane to above reaction mass. The reaction mixture was stirred for 10 hr at 25-30° C. The reaction mixture was filtered and washed with 100 mL heptane. The solid obtained was dried at 50° C. for 8 hr to obtain Stage C/Formula II. Dry wt.=64-68 g.

### Example 4: Alfaxalone Purification and Isolation

The crude alfaxalone purified by flash chromatography and following chromatographic condition.

### Mobile Phase A: Water.

### Mobile Phase B: Acetonitrile.

Flash chromatography column: C18, 120 g, 20-40 μm, 100 A°. Column oven temperature: room temperature.
After flash chromatography, all fractions of main peak are collected. The acetonitrile was distilled out under vacuum at 55° C. The residue was lyophilized. Isolated white solid. Arranged clean and dry 4-neck 500 mL RBF. Charged 55-60 g of above solid at RT. Charged 275 mL methanol at RT. The reaction mixture was stirred at 55° C. for 2h. The solution was passed through hyflow bed. Hyflow bed was washed with 55 mL methanol. The filtrate was distilled out under vacuum at 55° C. Charged 165 mL n-Heptane to above solid. Stirred for 15 min at RT and filtered. The solid was washed with 55 mL n-Heptane. The obtained solid was dried in air oven at 60° C. for 12 hr. Dry Wt.=55-60 g. Sample send for Complete analysis.

### Example 5: Stability Study of the Alfaxalone compound

Couple of batches of the Alfaxalone compound obtained by the process of the present invention were subjected to stability study at storage conditions of 30° C.±2° C. at 65%±5% RH and 40° C.±2° C. at 75%±5% RH for 6 months duration. The results are tabulated in Table I.

TABLE I

| Sl. No. | Test | Specification | Initial | 3 months | 6 months |
|---|---|---|---|---|---|
| 1 | Description | White to creamy white colour | white colour | white colour | white colour |
| | | powder | powder | powder | powder |
| 2 | Identification | Retention time of Principal peak | Complies | Complies | Complies |
| | by HPLC | in the sample solution should be | | | |
| | | correspond to that observed in | | | |
| | | the standard solution | | | |
| 3 | Loss on drying | Not more than 0.5 | Complies | Complies | Complies |
| | (% w/w) | | | | |
| 4 | Assay (On dried | Not less than 98.0 and not more | Complies | Complies | Complies |
| | Basis) (% w/w) | than 102.0 | | | |

Technical advantages of the process of the present invention over the prior arts are as following:
1. Simple, eco-friendly, cost effective and commercially viable process.
2. Avoids tough reaction conditions like Temperature and Reaction time.
3. Use of non-ionic compound i.e. (−)-diisopinocampheyl chloroborane as reducing agent.
4. Use of cheaper reagents and solvents. For example, reducing agent (−) DIP chloride as used in the present invention is cost effective in comparison to RuCl(S,S)-Tsdpenl(p-cymene)

1. A process for preparation of Alfaxalone compound of structural Formula I, comprising the steps of:
(a) reacting compound of structural Formula III with a reducing agent to give a mixture of diastereoisomeric compound of structural Formula II in an organic solvent at a temperature in the range of −30° C. to 50° C., wherein the reducing agent is organoboron compound, 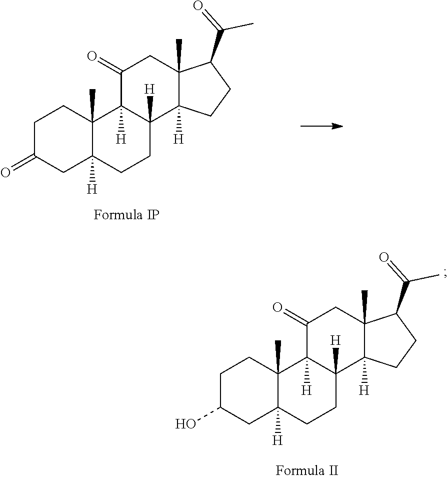
and
(b) separating diastereoisomeric compound of structural Formula II by chromatographic technique and then filtering and drying to get compound of structural Formula I:

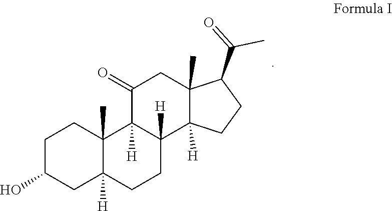

2. The process as claimed in claim 1, wherein the organoboron compound is selected from (−)-diisopinocampheyl chloroborane ((−) DIP chloride) 9-Borabicyclo[3.3.1]nonane (9-BBN), or diborane.

3. The process as claimed in claim 1, wherein the organic solvent is selected from tetrahydrofuran (THF) dichloromethane, diethyl ether, or methyl tertiary-butyl ether (MTBE).

4. The process as claimed in claim 1, wherein the step (a) is carried out at a temperature in the range of −10° C. to 0° C.

5. The process as claimed in claim 1, wherein the chromatographic technique is selected from flash chromatography, column chromatography, or liquid chromatography.

6. The process as claimed in claim 1, wherein the chromatographic technique is flash chromatography and in step (b) flash chromatography is used with a solvent at a mobile phase to give alfaxalone compound of structural Formula I.

7. The process as claimed in claim 1, wherein Formula III is prepared by a process, comprising the steps of:
(i) reacting compound of structural Formula V (1 la-Hydroxyprogesterone) with an oxidizing agent to get compound of structural Formula IV: 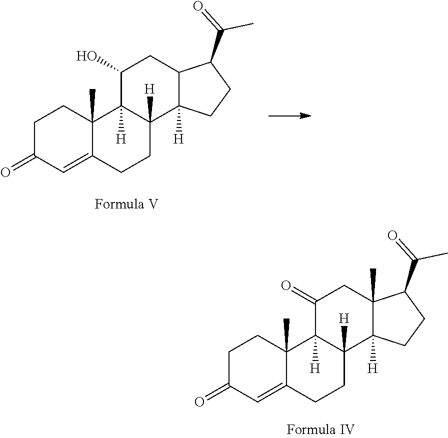
(ii) reacting compound of structural Formula IV with a reducing agent to get compound of structural Formula III:

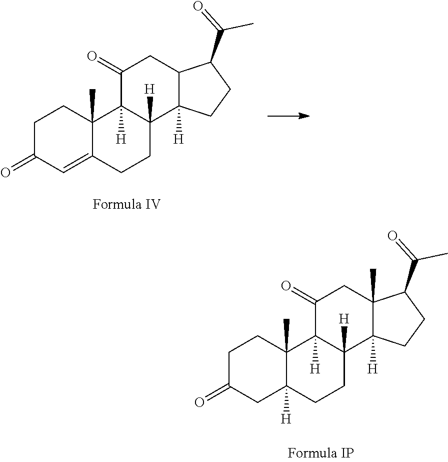

8. The process as claimed in claim 7, wherein the reaction of step (i) is carried out in presence of a solvent and the solvent is acetic acid and the oxidizing agent used in step (i) is selected from chromium trioxide, Manganese (IV) oxide, pyridinium chlorochromate, sodium dichromate, potassium dichromate, or DMSO-Oxalyl chloride (Swerm oxidation).

9. The process as claimed in claim 7, wherein the reaction of step (ii) is carried out in presence of a solvent and is selected from Dichloromethane (DCM), Isopropyl alcohol (IPA) and combination thereof and the reducing agent is 10% Palladium on carbon (10% Pd/C).

10. The process as claimed in claim 7, wherein the step (i) is carried out at a temperature in the range of 25-35° C. and the step (ii) is carried out at a temperature in the range of 50-55° C.

11. The process as claimed in claim 5, wherein the chromatographic technique is flash chromatography and in step (b) flash chromatography is used with a solvent at a mobile phase to give alfaxalone compound of structural Formula I.
